(19) United States
(12) Patent Application Publication
(10) Pub. No.: US 2008/0280363 A1
(43) Pub. Date: Nov. 13, 2008

(54) Pseudotyped Baculovirus and its Use

(76) Inventors: Kari Juhani Airenne, Kuopio (FI); Seppo Yla-Herttuala, Kuopio (FI); Minna U Kaikkonen, Kuopio (FI); Jani K. Raty, Kuopio (FI)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/666,847

(22) PCT Filed: Nov. 3, 2005
(86) PCT No.: PCT/GB2005/004263

§ 371 Date: Feb. 28, 2008

(30) Foreign Application Priority Data
Nov. 3, 2004 (GB) 0424357.2

(51) Int. Cl. C12N 7/00; C12N 15/86

(52) U.S. Cl. 435/456; 435/235.1

(57) ABSTRACT

A viral G protein-pseudotyped baculovirus, in which the G protein is truncated and comprises the ectodomain, transmembrane domain and cytoplasmic tail domain. Such a baculovirus can be used for transduction of cells and for gene therapy.

### FIELD OF THE INVENTION

This invention relates to a pseudotyped baculovirus and its use.

### BACKGROUND OF THE INVENTION

The use of baculovirus for gene therapy is of growing importance. Baculoviruses do not replicate in mammalian cells, are easy to manipulate and can house large foreign DNA inserts. They have been shown to transduce various cell types in vitro and in vivo with significant efficiency. However, efficient gene delivery still remains a challenge for baculovirus as for other gene therapy vectors in many target cells. Efficient transduction would mean high transgene expression with a lower viral load. Lower multiplicity of infection (MOI) should also decrease possible immune responses caused by the gene therapy vector by lowering the total dose administered.

A straightforward method to increase transduction efficiency is viral pseudotyping, which alters viral tropism by modifying, replacing or adding components to virus surface. These components are often derived from other viruses and may have specific or wide tropism (Verhoeyen & Cosset, 2004). One of the most widely used pseudotyping tools is the G glycoprotein of vesicular stomatitis virus (VSV-G); see, for example, Yun et al., 2003; Tang et al., 2001; Quinonez & Sutton, 2002; Barsoum et al., 1997. VSV-G pseudotyping is routinely used to enhance the target range and transduction efficiency of retroviruses (Emi et al., 1991; Burns et al., 1993; Mizuarai et al., 2001), by conferring augmented resistance to the complement inactivation and improved viral stability (Ory et al., 1996; Burns et al., 1993). Several reports of VSV-G-pseudotyped baculoviruses provide evidence that VSV-G is able to enhance transduction efficiency of baculovirus in vertebrate cells in vitro and in vivo (Barsoum et al., 1997; Park et al., 2001; Tani et al., 2003; Tani et al., 2001). It has been suggested that the improved transduction efficiency is a result of increase in the escape of baculovirus nucleocapsids from the endosomes (Barsoum et al., 1997; Park et al., 2001). VSV-G-pseudotyped baculovirus was very recently shown also to exhibit greater resistance to inactivation by animal sera than the WT baculovirus (Tani et al., 2003).

It has been shown that a VSV vector expressing a G protein ectodomain (G stem, GS) of 42 amino acids together with the transmembrane (TM) and cytoplasmic tail (CTD) domains confers efficient virus budding, probably by inducing membrane curvature at sites of virus assembly (Robison & Whitt, 2000). Recombinant viruses, having 12 or more membrane proximal residues, produced near wild-type levels of virus particles. Furthermore, it has been demonstrated that the same VSV-G fragment was able to induce hemifusion and potentiate the membrane fusion activity of some heterologous viral envelope proteins when the two proteins were coexpressed in BHK-21 cells (Jeetendra et al., 2002). Only 14 membrane-proximal residues in addition to TM/CTD were needed for the enhanced fusion activity of studied viral glycoproteins. It has also been demonstrated that the GS-region of VSV-G can be used as a membrane anchor in displaying fusion proteins on a baculovirus surface (Chapple & Jones, 2002; Ojala et al., 2004).

### SUMMARY OF THE INVENTION

The present invention is based on observations of a baculovirus vector which displays on its envelope a 21-amino acid ectodomain in addition to the TM and CTD domains of VSV-G, in its effect on the baculovirus infection and transduction rate in insect and vertebrate cells, respectively. The resulting virus was efficiently produced in high titers and resulted in several fold higher transduction efficiency in HeLa, SKOV-3, HepG2, 293T and BT4C cell lines as compared to control virus. Not only was the number of transduced cells increased but the cells showed higher levels of β-galactosidase activity. Increased transduction efficiency was also detected in rabbit muscle and rat brain in vivo.

A construct of the present invention thus provides improved baculovirus-mediated gene delivery, without compromising high viral titers. This strategy may prove to be useful also with other viral vectors, to aid gene delivery in vitro and in vivo.

### BRIEF DESCRIPTION OF THE DRAWING

FIG. 1 is a schematic representation of an embodiment of the invention.

### DESCRIPTION OF PREFERRED EMBODIMENTS

The combination of part or all of the ectodomain, the transmembrane and cytoplasmic tail domains represents a truncation of the G protein. Preferably, the pseudotyping is with those domains only, or with functional fragments thereof. It will be understood that the functionalities of other parts of the G protein can be avoided, and this may be responsible for the reduced cytotoxicity that is observed, relative to VSV-G pseudotyping.

For transduction, the baculovirus may include a foreign gene. The nature of this gene is not critical, and will be chosen having regard to its intended use. Thus, for example, a gene expressing a therapeutic protein may be chosen, e.g. to treat a brain-related, or spine-related or other injury.

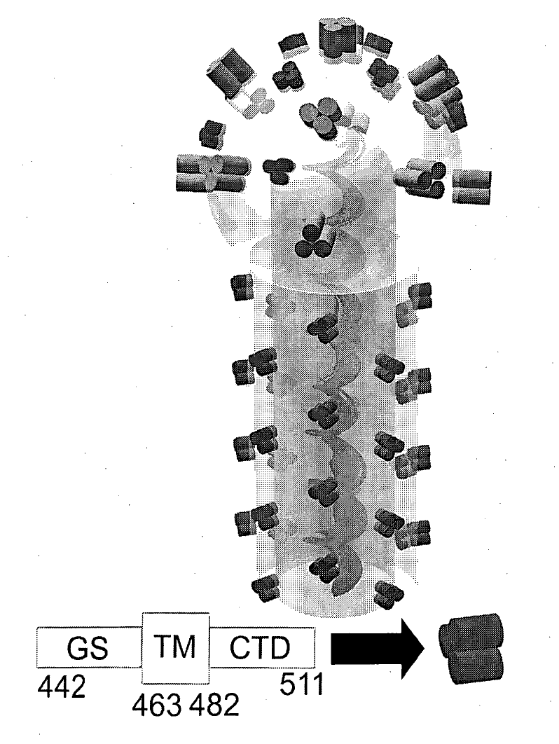

While the invention is defined by the claims, and others may be made and used in the same way or as would be evident to one skilled in the art, the illustrative embodiment, abbreviated herein as VSV-GED, is illustrated in FIG. 1. This schematic presentation of VSV-GED (indicated by arrow) pseudotyped baculovirus with numbers corresponding to the aminoacids of the VSV-G. The abbreviations used in the drawings are GS=G Stem, TM=Transmembrane domain, CTD=Cytoplasmic tail domain.

VSV-GED may be prepared as follows. The sequence encoding the 21 amino acid ectodomain together with TM/CTD of the VSV-GED was introduced into baculovirus genome under the strong polyhedrin promoter. VSV-GED display was confirmed from the concentrated viruses by immunoblotting using VSV-G antibody, which recognizes the 15 carboxy-terminal aminoacids of the VSV-GED. In agreement with a previous study (Robison & Whitt, 2000), trimer of VSV-GED was also detected on the immunoblot.

To examine the ratio of non-infective versus infective virus particles as compared to control baculovirus, immunoblotting with anti-vp39 and anti-gp64 was performed using gradient purified viruses. The result showed that the quantity of infective particles was consistent with the quantity of total particles. This indicates that the quality of the VSV-GS pseudotyped virus was comparable to that of the control virus. The titer of the concentrated virus was high (with 300× concentration 2,5*10E10), which suggests that VSV-GED pseudotyping does not disturb virus infectivity in insect cells.

The performance of the VSV-GS virus was studied in transduction assays using different cell lines. Even a modest increase in transduction efficiency is desirable, to decrease the possible systemic immune response and diminish the total dose administered. The increase in the transduction efficiency was remarkable in all studied cell lines excluding EAHY. EAHY cells have been previously shown to be resistant to baculovirus transduction (Kukkonen et al., 2003) and they showed no β-galactosidase expression even after VSV-GED virus treatment. Without wishing to be bound by theory, it seems that the block in the transduction of EAHY cells is not related to viral endosomal escape but rather to nuclear transport of the nucleocapsids. The difference in marker gene expression level was somewhat cell line-dependent, possibly reflecting variation in mechanisms associated with viral transduction.

When compared to the control virus, the VSV-GED pseudotyped virus resulted in higher transduction efficiency in all cell lines except EAHY, where only a neglible gene expression was detected. BT4C cells showed 75% transduction efficiency with the VSV-GED virus while the control virus transduced only 30% of the cells at MOI 50. Almost 15-fold increase in the transduction efficiency was observed at MOI 10. In general, the increase in the transduction efficiency was most prominent with low viral loads, i.e. MOIs under 200. With higher MOIs, the difference in the transduction efficiency diminished. However, in HepG2 cells, the difference was still notable at higher virus loads, MOI 200 resulting in an increase in the transduction efficiency from 20% to 70% and MOI 1000 from 60% to almost 100%.

The improved transduction was also evident when β-galactosidase enzyme activity was measured in the transduced cell lysates. Enzyme levels were increased almost 40-fold in BT4C cells at MOI 10. This difference was diminished at higher MOIs. However, in HepG2 cells, the difference was still clearly detectable with MOI 1000, in agreement with the above results relating to transduced cells. In 293T cells, the VSV-GED virus resulted in a six-fold increase in gene expression as compared to the control virus at MOI 10. HeLa showed a marked increase in β-galactosidase level compared to the control virus with MOIs 200 and 1000.

HeLa cells are often poorly permissive (Tani et al., 2001; Barsoum et al., 1997) for wild-type baculovirus, contrary to some reports showing effective transduction (Sarkis et al., 2000). The results reported here are in line with the findings that VSV-G protein is able to enhance the efficiency of transduction of several cell lines including HeLa. Further work suggests that VSV-GED allows earlier escape of the nucleocapsids after viral entry to the cell, which increases the nuclear transport of the viral genome and avoids lysosomal degradation of the viruses.

Rat brain and rabbit muscle were chosen for targets to study in vivo properties of the VSV-GED-pseudotyped baculovirus. The Rat model has previously been used to study the tropism of wild-type baculovirus (Lehtolainen et al., 2002), and the results of VSV-GED-pseudotyped virus were compared to this study. Interestingly, as the Lac-Z baculovirus transduces efficiently cuboid epithelium of choroid plexus cell and to some extent epithelial cells in brain microvessels, β-galactosidase staining after VSV-GED virus injection was mostly detected in the subarachnoid space and epithelial lining of the brain. Some enhancement in gene delivery was observed also in rabbit M. semimembranosus after intramuscular injection of VSV-GED-pseudotyped baculovirus. VSV-GED pseudotyping thus enhances baculovirus-mediated gene-delivery also in vivo.

VSV-GED pseudotyping can offer several advantages compared to the VSV-G pseudotyping of baculovirus. The small size of VSV-GED (8,6 kDa) should interfere less with viral infectivity and thus contribute favourably to gene delivery. The lack of syncytium formation (cytoxicity) of the infected cells during virus preparation should be beneficial for viral titers. VSV-GED infection resulted in significant syncytia formation only at pH 5.5, indicating that VSV-GED does not share the fusion properties of VSV-G. VSV-G has an extremely broad host range which is not desirable in targeted gene-delivery.

In conclusion, VSV-GED is able to aid baculovirus entry into vertebrate cells and provides a simple method to enhance baculovirus-mediated gene-delivery in vitro and in vivo. VSV-GED display has several advantages compared to VSV-G pseudotyping and may also provide a useful tool to augment gene-delivery of other vectors.

The following Example illustrates the invention.

Generation of the recombinant baculovirus. To remove gp64 and avidin sequences from Baavi (Raty et al., 2004), two linkers were introduced into the plasmid. A first linker (AAATAGATCTC-CTAGGAGATCTATTT) containing a BglII site was ligated to Swal I AvrII cut Baavi vector, in order to remove one of the three SmaI-sites. The two remaining SmaI-sites flanking the gp64 gene were used to remove this sequence from the vector. The removal of one of the three PstI sites resulted in two intact PstI sites flanking avidin sequence, enabling its elimination. The PstI site was removed by a combination of PstI partial digestion and SwaI digestion followed by ligation of a second linker (ATGCATTT B AAATGCATTGCA) containing a unique NsiI restriction site. The gene encoding VSV-G ectodomain (Chapple & Jones, 2002) was amplified with a 5′-primer GGGGTGATACTGGGCTATCCAA and a 3′-primer AGATCTTTACTTTCCAAGTCGGTTCA (BglII site underlined), and transferred into the SmaI site of vector. All steps were confirmed with restriction enzyme digestion.

Recombinant viruses were generated by using the Bac-to-Bac™ method according to manufacturer's instructions (Invitrogen) except for using more efficient E. coli strain in bacmid preparation (Airenne et al., 2003). Purification, concentration and titration of the virus particles were performed as described previously (Airenne et al., 2000). To verify the titer of the stock virus, end-point dilution was performed several times. Virus preparations were tested for sterility and analyzed for lipopolysaccharide and mycoplasma contamination.

Immunoblot analysis. Infected Sf9 cells (ATCC CRL-1711) and purified viruses were diluted 1:4 to sample buffer (0.125 M Tris HCl/pH 6.8/4% SDS/20% glycerol/0.004% bromophenol blue/10% 2-mercaptoethanol) and samples were denaturated at 100° C. for 10 min prior to SDS-PAGE and immunoblotting. Samples were loaded on reducing 10% sodium dodecyl sulphate-polyacrylamide gel. Molecular weight standard was supplied by Bio-Rad (Hercules, Calif., USA).

Samples were transferred onto a nitrocellulose membrane (Trans-Blot, Bio-Rad, USA) and the blots were probed with mouse anti-gp64 mAb (1:1000; Insight Biotechnology, Webley, UK), mouse anti-VSV-G (1:1000) or vp39 antibody (1:2000) as described previously (Laitinen et al., 2002). Finally, primary antibodies were detected with alkaline phosphatase-conjugated secondary antibodies (1:2000; Bio-Rad) followed by a colour reaction (NBT/BCIP, Roche, Basel, Switzerland).

Syncytium formation assay. This experiment was performed according to Zhang et al. (2003) except for the infection time, which was 48 h. Briefly, Sf9 cells were infected with LacZ (control), full VSV-G displaying virus similar to that described by Tani et al. (2003) or VSV-GED displaying virus at an MOI of 10. At 48 h post-infection, the growth medium (Insect-Xpress, Bio Whittaker) was removed, and cells were washed once with PBS at pH 7.4. The cells were then exposed to PBS at pH varying from 5.0 to 7.4 for 20 min. The PBS was removed and the cells were washed twice with PBS at pH 7.4 and returned to the growth medium. After 4 hours incubation at 28° C., cells were fixed with 1.25% glutaraldehyde in PBS for 20 min and examined for syncytia.
Transduction experiments. Cells were seeded at 7500 cells per well of 48-well plates (transduction efficiency) or 15000 cells per well of 24-well plates (quantitative assay) in their recommended medium. After 24 hours the medium was removed and fresh complete medium containing virus dilutions was added. Following 2 hr incubation at 37° C., 5% CO₂, 5 mM of sodium butyrate was added to all but HepG2 cells which had 2.5 mM of sodium butyrate. After 48 hr incubation, cells were stained with X-gal to visualize β-gal expressing cells (Thyronine et al., 1999) and blue cells were counted.
Endocytosis blocking. The experiment was performed as described by Kukkonen et al (2003). Briefly, cells were incubated in a medium supplemented with 0.5 μM monensin. 30 min later, viral dilutions (in medium containing 0.5 μM monensin) were added to the cells which were then incubated for 24 hours at 37° C. Finally cells were fixed with 1.25% glutaraldehyde and stained for LacZ activity.
Statistical analysis. Prism 4 from GraphPad was used to analyze the results with unpaired t-test to determine whether the differences between subgroups were statistically significant.
β-galactosidase enzyme assay. Luminescent beta-galactosidase enzyme assay (Clontech, BD Biosciences) was used to analyze the amount of enzyme expressed on the transduced cells according to the manufacturer's instructions. The luminescence was measured with black luminometer 96-well plates (Black Isoplate TC Wallac, Turku) and Victor 2 luminometer (Wallac, Turku).
Cytotoxicity assay. Cytotoxicity of Baavi was determined by an MTT-assay, CellTiter 96® Aqueous One Solution Cell Proliferation Assay (Promega), according to manufacturer's instructions. The measurements were done with a minimum of five replicates. Absorbance was measured at 492 nm. Survival percentage was calculated by comparing to the absorbance of the no virus or no butyrate wells (100% survival).
Protein concentration analysis. Coomassie Plus protein assay (Bio-Rad) was used to equalise the protein amounts from lysed cell samples, according to manufacturer's instructions.
Gene delivery into rat brain. Female inbred BDIX rats (200-250 g, n=7) were anaesthetized intraperitoneally with a solution (0.150 ml/100 g) containing fentanyl-fluanisone (Janssen-Cilag, Hypnorm®, Beerce, Belgium) and midazolame (Roche, Dormicum®, Basel, Switzerland), and placed into stereotaxic apparatus (Kopf Instruments). 2×10⁸ plaque-forming units of the virus vector in PBS/0.1% sucrose were injected by Hamilton syringe with a 27-gauge needle either into the right ventricle (coordinates: 1.0 mm caudal to bregma, 1.5 mm right to sutura sagittalis, and to a depth of 3.5 mm; n=3), or into the tumour of glioma-bearing rats (coordinates: 1.0 mm caudal to bregma, 1.0 mm right to sutura sagittalis, and to a depth of 2.5 mm; n=4). Rats were sacrificed on day 3 and perfused with PBS intracardially and fixed in X-gal fix for 30 min. After X-gal fix, brains were rinsed for 2 hours in PBS after which they were imbedded in OCT for later use.
Transduction of rabbit muscle. Rabbits (n=3) were anaesthetized with medetomidine-ketamine (Domitor 0.7 ml s.c; Orion Pharma, Espoo, Finland and Ketalar 0.9 ml s.c.; Pfizer, N.Y., USA). A total dose of 10⁹ pfu of either control baculovirus, with LacZ-marker gene, or VSV-GED baculovirus, was injected 10 times at a volume of 50 μl per M. semimembranosus muscle per injection. The rabbits were sacrificed 6 days after gene transfer and muscle samples were frozen in isopentane cooled with liquid nitrogen and stored at −70° C. The muscles were X-gal fixed (4% PFA in phosphate buffer, pH 7,2) for 30 min, washed in phosphate buffer for 2 hours and cryosectioned; X-gal staining was applied overnight in a humidified chamber at +37° C., followed by hematoxylin-eosin staining. For statistical analysis, 25 slides with two tissue sections in each slide were prepared from each animal. All positive cells were counted, areas were equalized and means were calculated.

### Generation and Characterization of VSV-GED Pseudotyped Baculovirus

Successful baculovirus production was studied by immunoblotting cell lysates and concentrated virus samples using gp64 antibody. Correct sized protein (˜64 kDa) was detected in all samples. The membrane incorporation of the VSV-GED was studied by immunoblotting using VSV-G antibody against the 15 carboxy-terminal amino acids (497-511) of VSV-G. The predicted size of the VSV-GED was 8.6 kDa and that sized protein was observed with VSV-G antibody in cell lysates and gradient purified concentrated virus samples. The amount of VSV-GED was comparable to gp64.

In order to determine the ratio of total particles to infective particles (tp/ip), immunoblotting with vp39 and gp64 antibody was performed and the results showed similar tp/ip ratio in VSV-GED to a wt-virus sample. The titers of the VSV-GED virus stocks were high, 2,5*10E10 pfu/ml (with 300× concentration), suggesting no adverse effect of VSV-GED to viral replication (infection) in insect cells.

To determine the pH requirement for viral membrane fusion, a syncytium formation assay was performed. Wild-type LacZ and full VSV-G pseudotyped baculoviruses were used as controls. No fusion activity was detected at pH>5.5 with the control virus or the VSV-GED virus. Infection with virus containing the entire VSV-G protein resulted in extensive syncytia formation during virus preparation in normal medium. The pH of the insect cell medium was 6.2.

Cytotoxicity of VSV-GED baculovirus was determined by Promega's MTT assay according to the manufacturer' instructions. No cytotoxicity was detected.

### Improved Transduction Efficiency In Vitro

The transduction efficiencies of HeLa, SKOV-3, Bt4C, HepG2, EAHY and 293T cells were determined with the VSV-GED and control virus using MOIs ranging from 10 to 1000. β-galactosidase staining was applied after transduction and the blue cells were counted to calculate the transduction efficiency.

The VSV-GED pseudotyped virus showed higher transduction efficiency in all cell lines except EAHY as compared to control virus. BT4C cells showed 75% transduction efficiency with VSV-GED virus while control virus transduced only 30% of the cells at MOI 50. Over 10-fold increase in the transduction efficiency was observed at MOI 10. In general, the increase in the transduction efficiency was most prominent with low viral load, i.e. MOI under 200. With higher MOIs, the difference in the transduction efficiency was diminished in BT4C and 293T. However, in HepG2, the difference was still detectable at MOI 1000, while MOI 200 resulted in a transduction increase from 20% to almost 70%.

In order to determine if the observed increase in transduction efficiency was also associated to an increase in the level of gene expression, β-galactosidase enzyme activity in the transduced cell lysates was measured. The results show that the enzyme-levels were increased almost 40-fold in BT4C cells at MOI 10. This difference was diminished at higher MOIs. However, with HepG2, the difference was still significant with MOI 1000 in agreement with the above transduction results. In 293T cells, VSV-GED virus resulted in a six-fold increase in gene expression rate as compared to control virus at MOI 10. HeLa showed marked increase in β-gal activities compared to LacZ virus with MOIs 200 and 1000. Monensin treatment prevented control virus transduction as measured by counting the blue cells, whereas VSV-GED transduction was partly retained in all the cell lines tested at the same timepoint.

### Improved Transduction In Vivo

Control LacZ baculovirus transduced efficiently cuboid epithelium of the choroid plexus and to some extent epithelial cells in brain microvessels. β-galactosidase expression after the VSV-GED virus injection was also detected in the walls of the lateral ventricles, subarachnoidal space and epithelial lining of the brain. The observed change in the transduced cells is in line with results obtained using VSV-G pseudotyped lentiviruses (Burns et al, 1993; Watson et al, 2002) and may indicate that VSV-GED fragment results in a transduction pattern similar to that of full-length VSV-G in vivo.

Viruses were injected also into New Zealand White rabbit M. semimembranosus and transduction was visualized by β-galactosidase staining. The control virus expression was concentrated near the injection site, while the VSV-GED displaying virus resulted in broader distribution of positive cells in muscle tissue. Modest transduction efficiency was detected in the rabbit skeletal muscle. Altogether, these results suggest that VSV-GED pseudotyping provides a simple mean to increase baculovirus transduction efficiency in vivo.

### REFERENCES

The following are incorporated herein by reference:
Airenne et al, 2000. Gene Therapy 7, 1499-1504.
Airenne et al, 2003. Nucleic Acids Res. 31, e101.
Barsoum et al, 1997. Hum. Gene Ther. 8, 2011-2018.
Burns et al, 1993. Proc. Natl. Acad. Sci. U.S.A. 90, 8033-8037.
Chapple & Jones, 2002. J. Biotechnol. 95, 269-275.
Emi et al, 1991. J. Virol. 65, 1202-1207.
Jeetendra et al, 2002. J. Virol. 76, 12300-12311.
Kukkonen et al, 2003. Mol. Ther. 8, 853-862.
Laitinen et al, 2002. Biochem. J. 363, 609-617.
Lehtolainen et al, 2002. Gene Therapy 9, 1693-1699.
Mizuarai et al, 2001. Biochem. Biophys. Res. Commun. 286, 456-463.
Ojala et al, 2004. Technol. Cancer Res. Treat. 3, 77-84.
Ory et al, 1996b. Proc. Natl. Acad. Sci. USA 93, 11400-11406.
Park et al, 2001. Biochem. Biophys. Res. Commun. 289, 444-450.
Quinonez & Sutton, 2002. DNA Cell Biol. 21, 937-951.
Raty et al, 2004. Mol. Ther. 9, 282-291.
Robison & Whitt, 2000. J. Virol. 74, 2239-2246.
Sarkis et al, 2000. Proc. Natl. Acad. Sci. U.S.A 97, 14638-14643.
Tang et al, 2001. J. Neurovirol. 7, 548-555.
Tani et al, 2003. J. Virol. 77, 9799-9808.
Tani et al, 2001. Virology 279, 343-353.
Thyronine et al, 1999. Gene Ther. 6, 6-11.
Verhoeyen & Cosset, 2004. J. Gene Med. 6 Suppl 1, S83-S94.
Watson et al, 2002. Mol. Ther. 5, 528-537.
Yun et al, 2003. Hum. Gene Ther. 14, 1643-1652.
Zhang et al, 2003. J. Virol. 77, 6265-6273.

1. A viral G protein-pseudotyped baculovirus, in which the G protein is truncated and comprises the ectodomain, transmembrane domain and cytoplasmic tail domain.

2. The baculovirus according to claim 1, wherein the ectodomain comprises 14 to 25 membrane-proximal residues.

3. The baculovirus according to claim 1, wherein the G protein is from vesicular stomatitis virus.

4. The baculovirus according to claim 1, which comprises a foreign gene.

5. A method for baculovirus-mediated transduction of target cells with a foreign gene, wherein said method comprises the use of a viral G protein-pseudotyped baculovirus, in which the G protein is truncated and comprises the ectodomain, transmembrane domain and cytoplasmic tail domain, and wherein the baculovirus further comprises a foreign gene.

6. (canceled)

7. The method, according to claim 5, wherein the ectodomain comprises 14 to 25 membrane-proximal residues.

8. The method, according to claim 5, wherein the G protein is from vesicular stomatitis virus.

9. A method for practicing gene therapy, wherein said method utilizes baculovirus-mediated transduction of target cells with a foreign gene, wherein said method comprises the use of a viral G protein-pseudotyped baculovirus, in which the G protein is truncated and comprises the ectodomain, transmembrane domain and cytoplasmic tail domain, and wherein the baculovirus further comprises a foreign gene.

10. The method, according to claim 9, wherein the ectodomain comprises 14 to 25 membrane-proximal residues wherein the G protein is from vesicular stomatitis virus.

11. The method, according to claim 9, wherein the G protein is from vesicular stomatitis virus.
